(19) United States
(12) Patent Application Publication
(10) Pub. No.: US 2024/0206807 A1
(43) Pub. Date: Jun. 27, 2024

(54) ELECTRONIC DEVICE, AND BIOMETRIC INFORMATION NOTIFICATION METHOD FOR ELECTRONIC DEVICE

(71) Applicant: SAMSUNG ELECTRONICS CO., LTD., Suwon-si (KR)

(72) Inventors: Hyogil KIM, Suwon-si (KR); Seongmin Je, Suwon-si (KR); Seongho Cho, Suwon-si (KR); Minhee Hwang, Suwon-si (KR)

(73) Assignee: SAMSUNG ELECTRONICS CO., LTD., Suwon-si (KR)

(21) Appl. No.: 18/600,203

(22) Filed: Mar. 8, 2024

Related U.S. Application Data
(63) Continuation of application No. PCT/KR22/12977, filed on Aug. 30, 2022.

(30) Foreign Application Priority Data
Sep. 30, 2021 (KR) 10-2021-0130125

(51) Int. Cl. A61B 5/00; A61B 5/145

(52) U.S. Cl. CPC A61B 5/486; A61B 5/14532; A61B 5/4809; A61B 5/4812; A61B 5/4866; A61B 5/681; A61B 5/7275; A61B 5/7282; A61B 5/742

(57) ABSTRACT

An electronic device includes a housing; a sensor module in the housing; a display; a memory; and a processor configured to monitor a blood glucose level of a user by using the sensor module; determine that the user is eating; record, in the memory, both a timepoint, of confirming that the blood glucose level exceeds the specific level, and the blood glucose level at the timepoint; determine, based on blood glucose records, a first timepoint, after the first meal, and a second timepoint, before the second meal; calculate an average value of blood glucose levels between the first timepoint and the second timepoint; obtain accumulated average value records daily; calculate a baseline blood glucose level of the user by using the plurality of accumulated average value records; and determine a low blood glucose reference level of the user based on the calculated baseline blood glucose level.

### CROSS-REFERENCE TO RELATED APPLICATIONS

This application is a by-pass continuation of International Application No. PCT/KR2022/012977, filed on Aug. 30, 2022, which is based on and claims priority to Korean Patent Application No. 10-2021-0130125, filed on Sep. 30, 2021, in the Korean Intellectual Property Office, the disclosures of which are incorporated by reference herein in their entireties.

### BACKGROUND

### 1. Field

The disclosure relates to an electronic device including a wearable device and, more particularly, to an electronic device and method for the electronic device to provide a notification related to biometric information.

### 2. Description of Related Art

Recently, a variety of electronic devices are being developed to measure biometric information of users and provide various health-related information based on the measured biometric information. For example, it is possible to determine whether there is an abnormality in a user's health by using the measured biometric information.

Additionally, various applications may be installed and executed on an electronic device. Applications may monitor the user's health status by periodically measuring the user's biometric information.

An electronic device may measure the user's biometric information and provide state information related to various diseases. For example, the electronic device may determine whether the user's blood glucose level corresponds to hypoglycemia. Hypoglycemia, especially nocturnal hypoglycemia that occurs during sleep, may lead to serious consequences such as hypoglycemic coma, cognitive dysfunction, neurological sequelae, or worsening cardiovascular disease. To prevent nocturnal hypoglycemia, it may be necessary to use insulin analogues that mimic the pattern of endogenous insulin secretion, consume a snack based on blood glucose levels before bedtime, and adjust the intensity and timing of evening exercise.

Non-invasive blood glucose monitoring methods using optical technology lack accuracy compared to invasive methods, so it may be difficult to determine and respond to a low blood glucose situation using absolute values.

Additionally, blood glucose levels and hypoglycemia criteria may vary from person to person, and determining hypoglycemia according to uniform criteria based on simple blood glucose levels or absolute values may fail to reflect individual characteristics.

Further, even for the same user, the presence or likelihood of hypoglycemia may vary depending on the situation (e.g., immediately after eating, immediately before falling asleep, during sleep, or during exercise), and determining hypoglycemia according to uniform criteria based on simple blood glucose levels or absolute values may fail to reflect these situations.

### SUMMARY

According to an aspect of the disclosure, an electronic device includes: a housing; a sensor module disposed inside the housing; a display; a memory; and a processor operably connected to the sensor module, the display, and the memory, wherein the processor is configured to: monitor a blood glucose level of a user by using the sensor module; determine that the user is eating based on confirming that the blood glucose level exceeds a specific level; record, in the memory, both a time point of confirming that the blood glucose level exceeds the specific level and the blood glucose level at the time point; determine, based on blood glucose records between a first meal and a second meal, a first time point past a first given time after the first meal and a second time point preceding a second given time before the second meal; determine an average value of blood glucose levels between the first time point and the second time point; obtain a plurality of accumulated average value records by accumulating the average value daily; determine a baseline blood glucose level of the user by using the plurality of accumulated average value records; and determine a low blood glucose reference level of the user based on the baseline blood glucose level.

According to an aspect of the disclosure, a method for an electronic device to notify biometric information, includes: monitoring, by using a sensor module, a blood glucose level of a user; based on confirming that the blood glucose level exceeds a specific level, determining that the user is eating; recording, in a memory, at least one of a time point of confirming that the blood glucose level exceeds a specific level or the blood glucose level at the time point; determining, based on blood glucose records between a first meal and a second meal, a first time point past a first given time after the first meal and a second time point preceding a second given time before the second meal; determining an average value of blood glucose levels between the first time point and the second time point; obtaining a plurality of average value records by accumulating the average value daily; determining a baseline blood glucose level of the user by using the plurality of average value records; and determining a low blood glucose reference level of the user based on the baseline blood glucose level of the user.

### Advantageous Effects of Invention

According to one or more embodiments, the electronic device and biometric information notification method thereof may perform non-invasive blood glucose monitoring using optical technology in a continuous or periodic manner, and define individual low blood glucose situations by observing baseline blood glucose and postprandial blood glucose in the user's designated contexts, which can compensate for the lack of accuracy in blood glucose measurement compared to invasive methods.

According to one or more embodiments, the electronic device and biometric information notification method thereof may calculate more accurate baseline blood glucose for each user by using a continuous or periodic scheme.

According to one or more embodiments, the electronic device and biometric information notification method thereof may predict the occurrence of hypoglycemia in a user and set a blood glucose measurement periodicity and notification output according to the user's biometric signals and movement information, which can enable the user to predict the occurrence of hypoglycemia in various situations and take immediate action against the low blood glucose situation.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram of an electronic device in a network environment according to ones or more embodiments;

FIG. 2A is a perspective views of an electronic device according to ones or more embodiments;

FIG. 2B is a perspective views of an electronic device according to ones or more embodiments;

FIG. 3 is an exploded perspective view of an electronic device according to ones or more embodiments;

FIG. 4 is a block diagram showing the configuration of an electronic device according to ones or more embodiments;

FIG. 5 illustrates a situation in which an electronic device measures a user's blood glucose level and displays a notification according to ones or more embodiments;

FIG. 6A is a graph depicting changes in blood glucose level after food intake according to ones or more embodiments;

FIG. 6B illustrates a method of calculating a user's baseline blood glucose level in the electronic device according to ones or more embodiments;

FIG. 6C illustrates a method of calculating a user's baseline blood glucose level in the electronic device according to ones or more embodiments;

FIG. 7A illustrates a part of a method in which the electronic device determines a user's exercise situation and provides a notification to prevent a low blood glucose state before and after exercise according to ones or more embodiments;

FIG. 7B illustrates a part of the method in which the electronic device determines the user's exercise situation and provides the notification to prevent the low blood glucose state before and after exercise according to ones or more embodiments

FIG. 8A illustrates part of a method in which the electronic device identifies a user's sleep status and provides a notification to prevent a low blood glucose state before and during sleep according to ones or more embodiments;

FIG. 8B illustrates part of the method in which the electronic device identifies the user's sleep status and provides a notification to prevent the low blood glucose state before and during sleep according to ones or more embodiments;

FIG. 8C illustrates part of the method in which the electronic device identifies the user's sleep status and provides a notification to prevent the low blood glucose state before and during sleep according to ones or more embodiments;

FIG. 9 is a diagram illustrating a situation in which an electronic device 400 and an external device 505 work together to provide a biometric information notification according to ones or more embodiments; and

FIG. 10 illustrates an operation of the electronic device to display a notification depending on individual situations according to ones or more embodiments.

### DETAILED DESCRIPTION

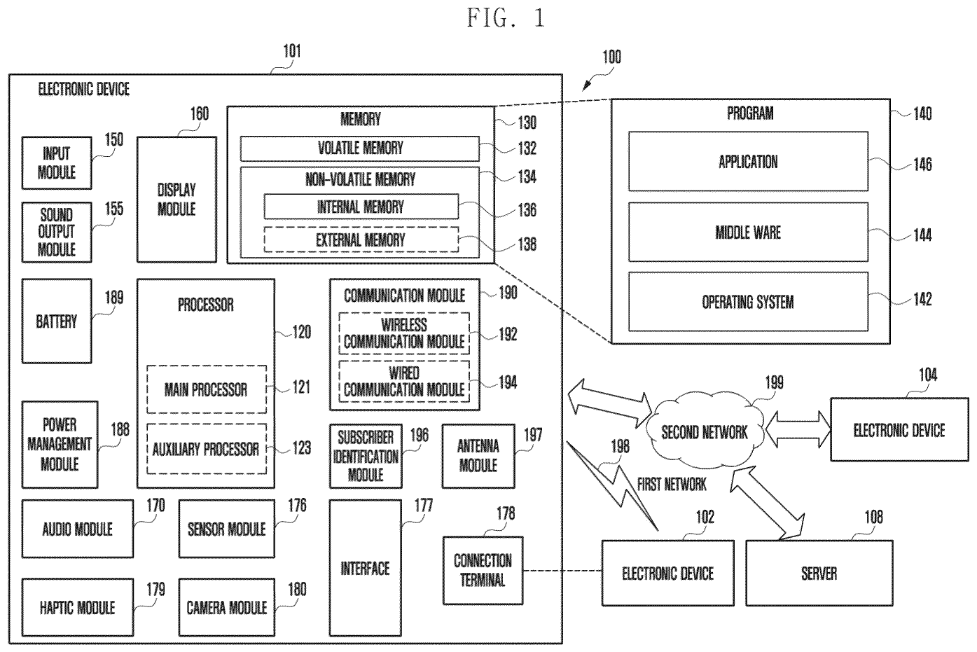

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mm Wave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IOT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

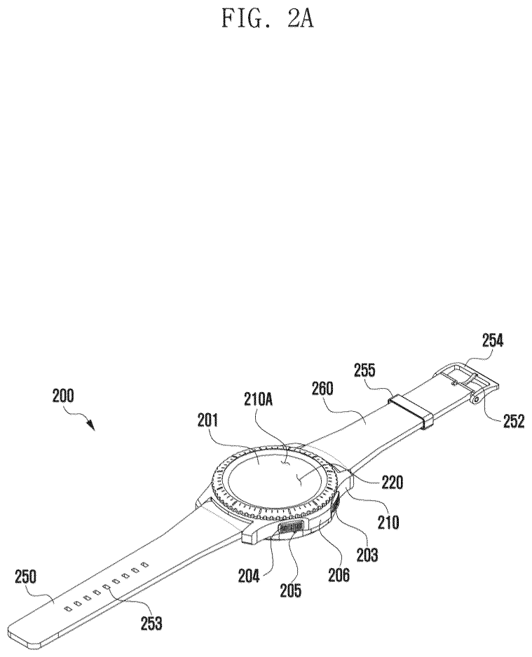

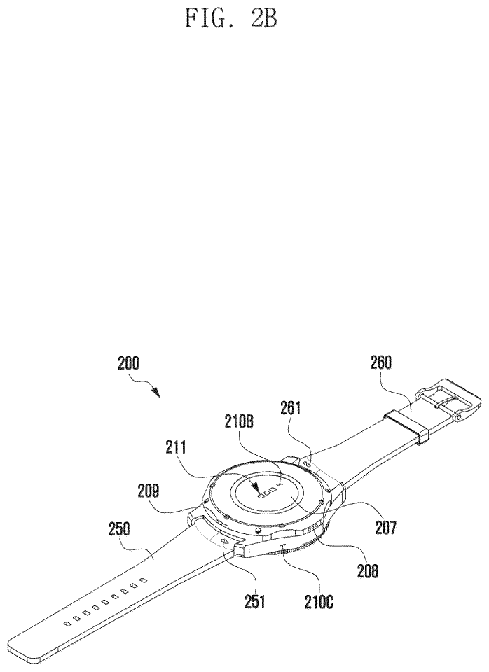

With reference to FIG. 2A and FIG. 2B, the electronic device 200 (e.g., electronic device 101 in FIG. 1) according to one or more embodiments may include: a housing 210 including a first surface (or, front surface) 210A, a second surface (or, rear surface) 210B, a side surface 210C surrounding the space between the first surface 210A and the second surface 210B, fastening member 250 and fastening member 260 connected to at least a portion of the housing 210 and configured to detachably fasten the electronic device 200 to a body part (e.g., wrist, ankle, etc.) of the user. According to one or more embodiments, the housing may refer to a structure forming some of the first surface 210A, the second surface 210B, and the side surface 210C in FIG. 2A. According to one or more embodiments, the first surface 210A may be formed by a front plate 201 that is substantially transparent at least in part (e.g., glass plate containing various coating layers, or polymer plate). The second surface 210B may be formed by a rear plate 207 that is substantially opaque. The rear plate 207 may be made of, for example, coated or colored glass, ceramic, polymer, metal (e.g., aluminum, stainless steel (STS), or magnesium), or a combination thereof. The side surface 210C is coupled to the front plate 201 and the rear plate 207 and may be formed by a lateral bezel structure (or, lateral member) 206 containing metal and/or polymer. According to one or more embodiments, the rear plate 207 and the lateral bezel structure 206 may be integrally formed and contain the same material (e.g., metal material such as aluminum). The fastening member 250 and the fastening member 260 may be made of various materials and formed in various shapes. The fastening member 250 and the fastening member 260 may be formed as a single body or as plural unit links that are movable with each other, by woven material, leather, rubber, urethane, metal, ceramic, or a combination thereof.

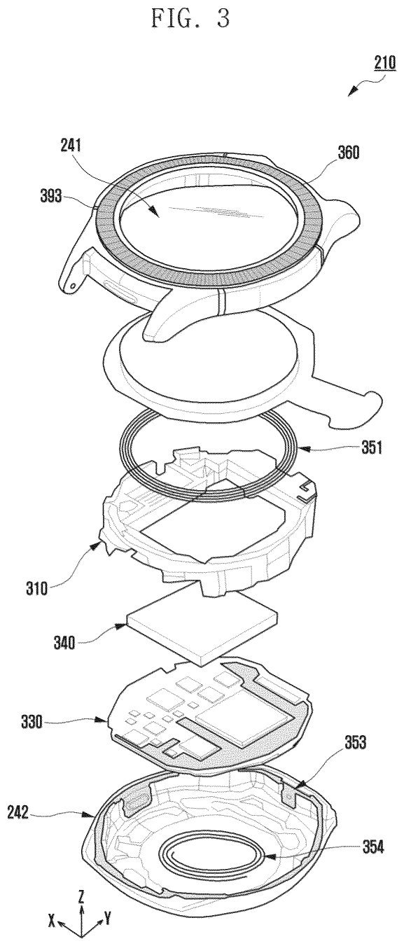

According to ones or more embodiments, the electronic device 200 may include at least one of a display 220 (refer to FIG. 3), an audio module (e.g., audio module 205 and audio module 208), a sensor module 211, key input device 202, key input device 203 and key input device 204, or a connector hole 209. According to one or more embodiments, at least one of the components (e.g., any of key input device 202, key input device 203 and key input device 204, connector hole 209, and sensor module 211) may be removed from the electronic device 200, or a different component may be added to the electronic device 200.

The display 220 can be exposed through, for example, a significant portion of the front plate 201. The display 220 may have a shape corresponding to the shape of the front plate 201 and may have one of various shapes such as a circle, an ellipse, and a polygon. The display 220 may be disposed in combination with or adjacent to a touch sensing circuit, a pressure sensor capable of measuring the intensity (pressure) of a touch, and/or a fingerprint sensor.

The audio module (e.g., audio module 205 and audio module 208) may include a microphone hole 205 and a speaker hole 208. In the microphone hole 205, a microphone for picking up external sounds may be disposed therein, and plural microphones may be arranged to sense the direction of sound according to one or more embodiments. The speaker hole 208 can be used for an external speaker and a call receiver. According to one or more embodiments, the speaker hole 208 and the microphone hole 205 may be implemented as a single hole, or a speaker (e.g., piezo speaker) may be included without the speaker hole 208.

The sensor module 211 may generate an electrical signal or data value corresponding to an internal operating state of the electronic device 200 or an external environmental state. The sensor module 211 may include, for example, a biometric sensor module 211 (e.g., HRM sensor) disposed on the second surface 210B of the housing 210. The electronic device 200 may further include a sensor module including at least one of, for example, a gesture sensor, a gyro sensor, an air pressure sensor, a magnetic sensor, an acceleration sensor, a grip sensor, a color sensor, an infrared (IR) sensor, a biometric sensor, a temperature sensor, a humidity sensor, or an illuminance sensor.

The sensor module 211 may include electrode region 213 and electrode region 214 that constitute some of the surface of the electronic device 200, and a biometric signal detection circuit electrically connected to the electrode region 213 and electrode region 214. For example, the electrode region 213 and electrode region 214 may include a first electrode region 213 and a second electrode region 214 disposed on the second surface 210B of the housing 210. The sensor module 211 may be configured such that the electrode region 213 and electrode region 214 obtain electrical signals from a body part of the user and the biometric signal detection circuit detects the user's biometric information based on the electrical signals.

The key input device 202, key input device 203, and key input device 204 may include a wheel key 202 disposed on the first surface 210A of the housing 210 and rotatable in at least one direction, and/or side key buttons 203 and 204 disposed on the side surface 210C of the housing 210. The wheel key may have a shape corresponding to the shape of the front plate 201. In another embodiment, the electronic device 200 may not include some or all of the key input device 202, key input device 203, and key input device 204 described above, and the key input device 202, key input device 203, and key input device 204 that is not included may be implemented in other forms, such as soft keys, on the display 220. The connector hole 209 may accommodate a connector (e.g., USB connector) for transmitting and receiving power and/or data to and from an external electronic device, and may include another connector hole that can accommodate a connector for transmitting and receiving an audio signal to and from an external electronic device. The electronic device 200 may further include, for example, a connector cover that covers at least a portion of the connector hole 209 and blocks foreign substances from entering the connector hole.

The fastening member 250 and the fastening member 260 may be detachably fastened to at least a portion of the housing 210 by using locking members 251 and 261. The fastening member 250 and the fastening member 260 may include one or more of a fixing member 252, fixing member fastening holes 253, a band guide member 254, and a band fixing ring 255.

The fixing member 252 may be configured to fix the housing 210 and the fastening member 250 and the fastening member 260 to a body part (e.g., wrist, or ankle) of the user. The fixing member fastening holes 253 may fix the housing 210 and the fastening member 250 and the fastening member 260 to a body part of the user in correspondence to the fixing member 252. The band guide member 254 may be configured to limit the range of movement of the fixing member 252 when the fixing member 252 engages with a fixing member fastening hole 253, so that the fastening member 250 and the fastening member 260 may be fastened in close contact to a body part of the user. The band fixing ring 255 may limit the range of movement of the fastening member 250 and the fastening member 260 while the fixing member 252 and the fixing member fastening hole 253 are fastened.

With reference to FIG. 3, the housing 210 (e.g., electronic device 101 in FIG. 1 or electronic device 200 in FIG. 2) may include a lateral bezel structure 310, a wheel key 320, a front plate 201, a display 241, a first antenna 351, a second antenna 354, a support member 360 (e.g., bracket), a battery 340, a printed circuit board 330, a sealing member 353, a rear plate 242, and fastening member 395 and fastening member 397. At least one of the components of the electronic device 330 may be identical or similar to at least one of the components of the electronic device 200 of any of FIG. 1 an FIG. 2, and repeated descriptions are omitted herein. The support member 360 disposed inside the electronic device 300 may be formed to be connected to the lateral bezel structure 310 or be integrally formed with the lateral bezel structure 310. The support member 360 may be made of, for example, a metal material and/or a non-metal (e.g., polymer) material. The support member 360 may have one surface coupled to the display 241 and the other surface coupled to the printed circuit board 380. A processor, a memory, and/or an interface may be mounted on the printed circuit board 380. The processor may include one or more of, for example, a central processing unit, an application processor, a graphics processing unit (GPU), a sensor processor, or a communication processor.

The memory may include, for example, a volatile memory or a nonvolatile memory. The interface may include, for example, a high definition multimedia interface (HDMI), a universal serial bus (USB) interface, an SD card interface, and/or an audio interface. The interface allows the electronic device 300 to electrically or physically connect to an external electronic device, and may include, for example, a USB connector, an SD card/MMC connector, or an audio connector.

The battery 340 is a device for supplying power to at least one component of the electronic device 300, and may include, for example, a non-rechargeable primary cell, a rechargeable secondary cell, or a fuel cell. At least a portion of the battery 370 may be disposed substantially on the same plane as, for example, the printed circuit board 380. The battery 370 may be disposed as a single body within the electronic device 300 or may be detachably disposed from the electronic device 300.

The first antenna 351 may be disposed between the display 241 and the support member 360. The first antenna 351 may include, for example, a near field communication (NFC) antenna, a wireless charging antenna, and/or a magnetic secure transmission (MST) antenna. For example, the first antenna 351 may perform short-range communication with an external device, wirelessly transmit or receive power required for charging, and transmit a short-range communication signal or a magnetic-based signal including payment data. In another embodiment, an antenna structure may be formed by some or a combination of the lateral bezel structure 310 and/or the support member 360.

The second antenna 354 may be disposed between the printed circuit board 330 and the rear plate 242. The second antenna 354 may include, for example, a near field communication (NFC) antenna, a wireless charging antenna, and/or a magnetic secure transmission (MST) antenna. For example, the second antenna 354 may perform short-range communication with an external device, wirelessly transmit or receive power required for charging, and transmit a short-range communication signal or a magnetic-based signal including payment data. In another embodiment, an antenna structure may be formed by some or a combination of the lateral bezel structure 310 and/or the rear plate 242.

The sealing member 353 may be positioned between the lateral bezel structure 310 and the rear plate 242. The sealing member 353 may be configured to block moisture and foreign matter flowing into the space surrounded by the lateral bezel structure 310 and the rear plate 353 from the outside.

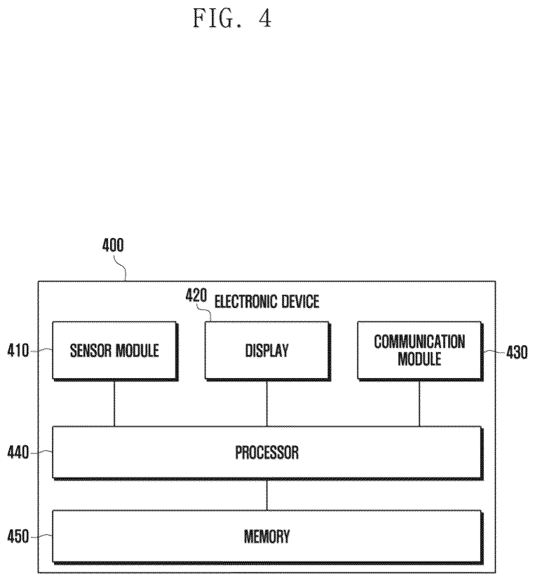

FIG. 4 is a block diagram showing the configuration of an electronic device according to ones or more embodiments.

With reference to FIG. 4, the electronic device 400 may include at least one sensor module 410, a display 420, a communication module 430, a processor 440, and/or a memory 450. The electronic device 400 may include at least some of the components and/or functions of the electronic device 101 of FIG. 1 and the electronic device 200 of FIG. 2 and/or FIG. 3. At least some of the components of the electronic device shown may be operably, functionally, and/or electrically connected to each other.

According to ones or more embodiments, the sensor module 410 may sense the operating state (e.g., power or temperature) of the electronic device 400 or the external environmental state (e.g., user state), and generate an electrical signal or data value corresponding to the sensed state. According to ones or more embodiments, the sensor module 410 may include, for example, a photoplethysmography sensor (PPG sensor), an angle sensor, a gyro sensor, a magnetic sensor, an acceleration sensor, a proximity sensor, or an illuminance sensor.

According to ones or more embodiments, the electronic device 400 may be referred to as a portable device such as a smartphone or a wearable device such as a smart watch or smart band. The user may estimate biometric information including at least one of, for example, heart rate, oxygen saturation (SpO₂), stress, or blood pressure by using a PPG sensor included in the electronic device 400. Hereinafter, the obtained biometric information will be explained by using blood glucose levels as an example. However, without being limited thereto, all biometric information that can be extracted from the PPG signal may be included.

According to ones or more embodiments, the display 420 may display various images under the control of the processor 440. The display 420 may be implemented with one of, but not limited to, liquid crystal display (LCD), light-emitting diode (LED) display, or organic light-emitting diode (OLED) display. The display 420 may be formed as a touchscreen that detects a touch and/or proximity touch (or hovering) input by using a user's body part (e.g., finger) or input device (e.g., stylus pen). The display 420 may include at least some of the components and/or functions of the display module 160 in FIG. 1.

According to ones or more embodiments, the communication module 430 may communicate with an external device through a wireless network under the control of the processor 440. The communication module 430 may include hardware and software modules for transmitting and receiving data through a cellular network (e.g., long term evolution (LTE) network, 5G network, new radio (NR) network) or a short-range network (e.g., Wi-Fi, Bluetooth, Bluetooth low energy). The communication module 430 may include at least some of the components and/or functions of the communication module 190 in FIG. 1.

According to ones or more embodiments, the processor 440 is a constituent capable of performing operations or data processing related to control and/or communication of individual components of the electronic device 400, and may be composed of one or more processors. The processor 440 may include at least some of the components and/or functions of the processor 120 in FIG. 1.

According to ones or more embodiments, there will be no limitations to the calculation and data processing functions that the processor 440 can implement on the electronic device 400, but hereinafter features related to the operation of measuring biometric information using a sensor module and providing a biometric information notification will be described in detail. The operation of the processor 440 may be executed by loading instructions stored in the memory 450.

According to ones or more embodiments, the electronic device 400 may include at least one memory 450, and the memory 450 may include a main memory and a storage. The main memory may be composed of a volatile memory such as dynamic random access memory (DRAM), static RAM (SRAM), or synchronous dynamic RAM (SDRAM). The storage may include at least one of one time programmable ROM (OTPROM), PROM, EPROM, EEPROM, mask ROM, flash ROM, flash memory, hard drive, or solid state drive (SSD). Alternatively, the memory 450 may include a large capacity storage device as a non-volatile memory. For example, the memory 450 may include at least one of one time programmable ROM (OTPROM), PROM, EPROM, EEPROM, mask ROM, flash ROM, flash memory, hard drive, or solid state drive (SSD). The memory 450 may store various file data, and the stored file data can be updated according to the operation of the processor 440.

According to ones or more embodiments, the processor 440 may monitor the blood glucose level of the user by using the sensor module 410, present, if the blood glucose level exceeds a specific level, a guide screen asking whether the user has a meal, record the meal time and blood glucose level on the memory 450, determine, based on blood glucose records between a first meal and a second meal, a first time point past a given time after the first meal and a second time point preceding a given time before the second meal, calculate the average value of blood glucose levels in the section between the first time point and the second time point, calculate the baseline blood glucose level of the user by using a plurality of average value records, and determine a low blood glucose reference level of the user based on the calculated baseline blood glucose level. This may be called a continuous blood glucose measurement method. Alternatively, the processor 440 may monitor the blood glucose level of the user by using the sensor module 410, periodically present a guide screen to the user at a specific time to allow the user to select whether the user is eating, measure, upon confirming that the user is eating, the blood glucose level at regular intervals from a time point past a given time after the corresponding time point, calculate the baseline blood glucose level of the user based on the average value of the measured blood glucose levels, and determine a low blood glucose reference level of the user based on the calculated baseline blood glucose level. This may be named a periodic blood glucose measurement method.

According to ones or more embodiments, if the user's blood glucose level is below the low blood glucose reference level, the processor 440 may determine that the user is in a low blood glucose state. The user's low blood glucose reference level may correspond to approximately 60% of the calculated baseline blood glucose level of the user. Here, the figure of approximately 60% is not fixed and may vary depending on settings.

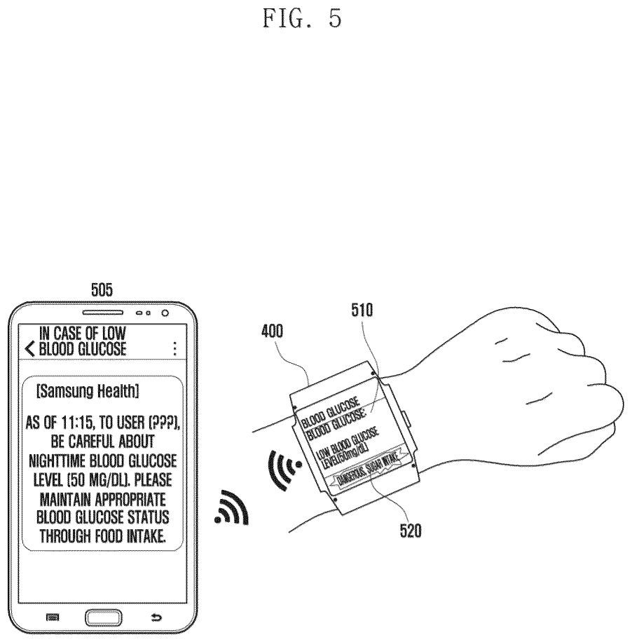

FIG. 5 illustrates a situation in which an electronic device measures a user's blood glucose level and displays a notification according to ones or more embodiments.

With reference to FIG. 5, the electronic device 400 may be in the form of a smartwatch and may include a gadget that measures the user's blood glucose level through a contact with the user's wrist or skin. For example, the electronic device 400 may measure the concentration of glucose in sweat secreted from the user's skin or interstitial fluid within the skin, and determine the user's blood glucose level based on the measured concentration of glucose. The electronic device 400 may display the measured blood glucose level 510 of the user. In this case, the electronic device 400 may display a warning message 520 indicating a low blood glucose state when the user's blood glucose level is below the reference range. The reference range of blood glucose may indicate the normal blood glucose level of a typical person. The reference range of blood glucose may include, for example, 80 to 160 mg/dL.

In addition, the electronic device 400 may establish a short-range wireless communication connection with an external device 505. The electronic device 400 may display information about the user's blood glucose state through the external device 505 by using the established short-range wireless communication connection. The electronic device 400 may also transmit not only the user's blood glucose level but also the measured glucose concentration and the rate of change in glucose concentration to the external device 505. The external device 505 may display the user's blood glucose level received from the electronic device 400. In this case, the external device 505 may display a warning message indicating a low blood glucose state if the received user's blood glucose level is below the reference range of blood glucose.

Further, the electronic device 400 may include a sensor module (e.g., sensor module 410 in FIG. 4) (e.g., motion sensor, body temperature sensor, electrocardiogram sensor, PPG sensor, GSR sensor, or pedometer). The electronic device 400 may use the sensor module 410 to obtain information about the user's blood glucose level, amount of exercise, body temperature, whether the user is in a sleeping state, hormonal cycle, or heart rate, and may determine whether the user's blood glucose level is within the low blood glucose range based on the obtained information about the user's blood glucose level, amount of exercise, body temperature, whether the user is in a sleeping state, hormonal cycle, or heart rate.

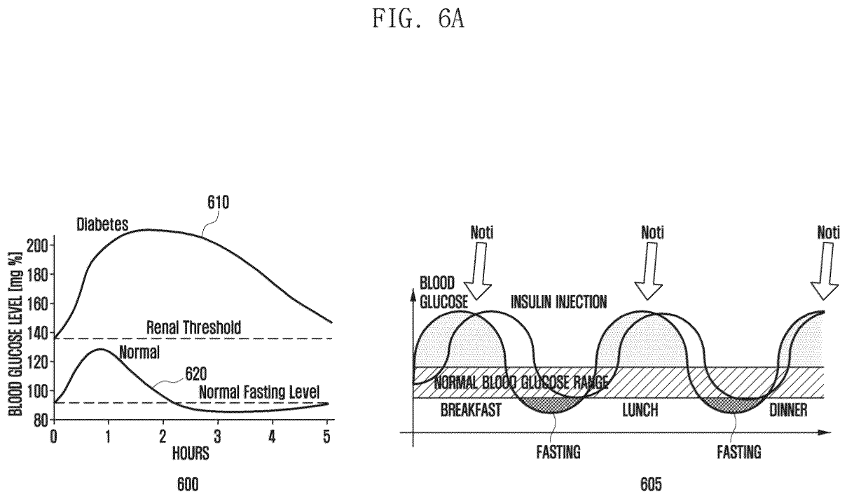

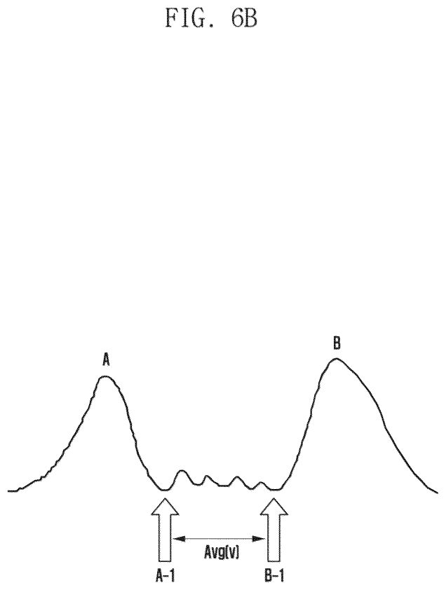

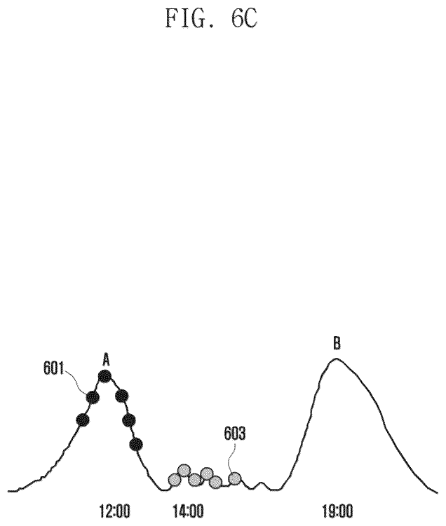

FIG. 6A is a graph depicting changes in blood glucose level after food intake according to ones or more embodiments. The electronic device 400 according to ones or more embodiments may measure the user's blood glucose level by using a sensor module (e.g., sensor module 410 in FIG. 4) and represent changes in blood glucose level over time as a graph (hereinafter, blood glucose curve 600). In the blood glucose curve 600, the horizontal axis may represent time, and the vertical axis may represent the blood glucose level in the user's body. The blood glucose curve 600 may include a first graph 610 and a second graph 620. The first graph 610 may indicate a graph of blood glucose levels before and after a meal for a diabetic patient. The second graph 620 may represent a graph of blood glucose levels before and after a meal for a normal non-diabetic person.

With reference to FIG. 6A, it can be seen that the blood glucose level of a diabetic patient on the first graph 610 is about 135 mg/dL or more, while the blood glucose level of a normal person on the second graph 620 is about 90 mg/dL.

In FIG. 6A, it can be seen that in the first graph 610, after a change in the impedance signal of the user's body is detected, the amplitude of the blood glucose curve 600 increases, and after about 2 hours, the blood glucose curve 600 reaches the maximum amplitude. Additionally, the first graph 610 shows that the amplitude of the blood glucose curve 600 gradually decreases as time passes after about 3 hours. In FIG. 6A, it can be seen that in the second graph 620, after a change in the impedance signal of the user's body is detected, the amplitude of the blood glucose curve 600 increases, and after about 40 minutes, the blood glucose curve 600 reaches the maximum amplitude. Additionally, the second graph 620 shows that the amplitude of the blood glucose curve 600 decreases relatively quickly as time passes after about 1 hour. A relatively rapid decrease in the amplitude of the blood glucose curve 600 may mean a situation in which the user's blood glucose level rapidly decreases due to the influence of insulin. A relatively gradual decrease in the amplitude of the blood glucose curve 600 may indicate a situation where insulin is insufficient or is not functioning properly.

In FIG. 6A, part 605 is a graph showing the blood glucose level and insulin level before and after a meal for a normal person. Referring to part 605, it may be seen that a normal person's blood glucose level reaches the highest level immediately after a meal, and when the blood glucose level reaches the highest level, the insulin level increases, which lowers the blood glucose level back to the normal range. Thereafter, it may be seen that the blood glucose level drops below the normal range during the fasting period between meals. If there is a lack of insulin, the blood glucose level may remain high without falling after a meal.

FIG. 6B illustrates a first method of calculating a user's baseline blood glucose level in the electronic device according to ones or more embodiments.

According to ones or more embodiments, the processor 440 may monitor the blood glucose level of the user by using the sensor module 410, present, if the blood glucose level exceeds a specific level, a guide screen asking whether the user has a meal, record the meal time and blood glucose level on the memory 450, determine, based on blood glucose records between a first meal and a second meal, a first time point past a given time after the first meal and a second time point preceding a given time before the second meal, calculate the average value of blood glucose levels in the section between the first time point and the second time point, calculate the baseline blood glucose level of the user by using a plurality of average value records, and determine a low blood glucose reference level of the user based on the calculated baseline blood glucose level. The baseline blood glucose level may refer to a blood glucose level that are lowered when insulin is secreted after a meal. This may vary depending on the individual's level of insulin secretion and eating habits.

In ones or more embodiments, the first method for obtaining the user's baseline blood glucose level may refer to a scheme that determines, based on blood glucose records between a first meal and a second meal, a first time point past a given time after the first meal and a second time point preceding a given time before the second meal, calculate the average value of blood glucose levels in the section between the first time point and the second time point, and calculate the baseline blood glucose level of the user by using a plurality of average value records.

The graph in FIG. 6B may represent the user's blood glucose level measured by the electronic device. Typically, the blood glucose level may rise after a meal and return to the normal range over time. The blood glucose level may rise the most within about an hour immediately after the first meal (A), and may return to the normal level about 2 to 3 hours after the meal as insulin is secreted. Thereafter, the blood glucose level may rise the most again within about an hour immediately after the second meal (B). The interval average between the lowest blood glucose level (A-1) closest to the first meal (A) and the lowest blood glucose level (B-1) closest to the second meal (B) before the second meal (B) is calculated. By repeating this operation for three or more days, candidates for the baseline blood glucose level can be selected.

The blood glucose level may increase immediately after a meal and then decrease over time. However, individual figures may vary depending on the user's characteristics and circumstances. For example, for a patient with diabetes, unlike a normal person without diabetes, the absolute value of the lowest blood glucose level (A-1) closest to the first meal (A) may be different from the absolute value of the lowest blood glucose level (B-1) closest to the second meal (B) before the second meal (B). Ones or more embodiments according to this document can complement the accuracy of non-invasive blood glucose measurement technology by determining the baseline blood glucose level in consideration of individual characteristics of the user.

FIG. 6C illustrates a second method of calculating a user's baseline blood glucose level in the electronic device according to ones or more embodiments.

According to ones or more embodiments, the processor 440 may monitor the blood glucose level of the user by using the sensor module 410, periodically present a guide screen to the user at a specific time to allow the user to select whether the user is eating, measure, upon confirming that the user is eating, the blood glucose level at regular intervals from a time point past a given time after the corresponding time point, calculate the baseline blood glucose level of the user based on the average value of the measured blood glucose levels, and determine a low blood glucose reference level of the user based on the calculated baseline blood glucose level.

In ones or more embodiments, the second method for obtaining the user's baseline blood glucose level may refer to a scheme that measures, upon confirming that the user is eating, the blood glucose level at regular intervals from a time point past a given time after the corresponding time point, and calculates the baseline blood glucose level of the user based on the average value of the measured blood glucose levels.

The graph in FIG. 6C may represent the user's blood glucose level measured by the electronic apparatus 400. Typically, the blood glucose level may rise after a meal and return to the normal range over time. The blood glucose level may rise the most within about an hour immediately after the first meal (A), and may return to the normal level about 2 to 3 hours after the meal as insulin is secreted. Thereafter, the blood glucose level may rise the most again within about an hour immediately after the second meal (B).

The processor 440 may present a guide screen asking whether the user is eating in response to a user input for eating or a meal time entered in advance if present. The processor 440 may confirm that the user is eating based on a user input by using an application on the electronic device 400 or an application on an external device connected to the communication module 430. The processor 440 may measure the blood glucose level at regular intervals (e.g., once every 10 minutes) for a given period of time (e.g., 60 minutes) from the time point when user's eating is confirmed. For example, the processor 440 may measure the blood glucose level a total of six times, once every 10 minutes, for 60 minutes from the time point when user's eating is confirmed. These six blood glucose measurement points before and after a meal may be named high blood glucose points 601 on the graph.

The processor 440 may measure the blood glucose level a total of six times, once every 10 minutes, for 60 minutes after a given period of time (e.g., 2 hours) from the time point when high blood glucose is confirmed. These six blood glucose measurement points past a given period of time after a meal may be named baseline blood glucose points 603 on the graph. The processor 440 may calculate the user's baseline blood glucose level by averaging the values of the baseline blood glucose points 603.

The processor 440 may calculate the user's personalized baseline blood glucose level by using the first method and/or the second method. The processor 440 may calculate the user's low blood glucose range by calculating the range of relative deviation based on this baseline blood glucose level. The user's low blood glucose range may be determined by multiplying the personalized baseline blood glucose level by a specific parameter (e.g., 0.6).

TABLE 1

| | | Baseline blood glucose |
|---|---|---|
| Classification | Normal range | range (+−20%) |
| Fasting blood glucose | 70~100 mg/dL | 85 +− 15 mg/dL |
| Blood glucose one hour | 90~140 mg/dL | 115 +− 25 mg/dL |
| after meal | | |

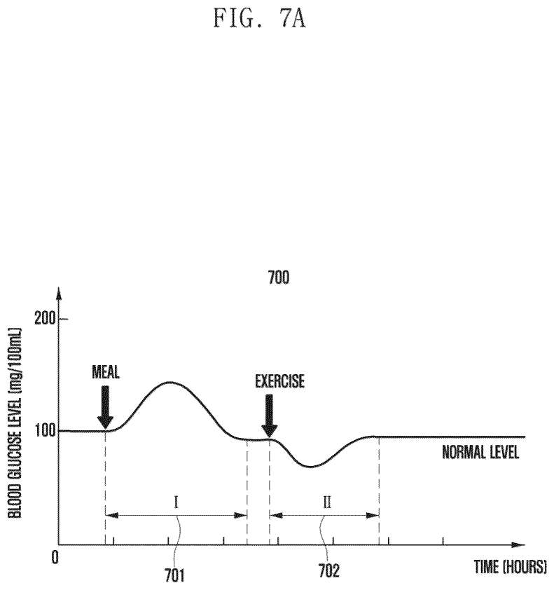

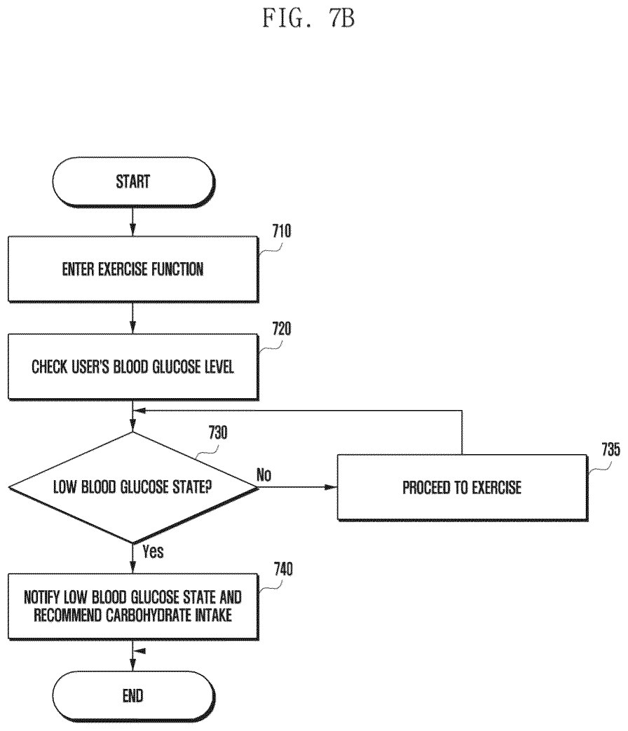

For example, if the user's baseline blood glucose is measured at 85 mg/dL during fasting, the processor 440 may determine a fasting low blood glucose range (e.g., 70 to 100 mg/dL) based on 85 mg/dL. Additionally, if the user's baseline blood glucose is measured at 115 mg/dL immediately after a meal, the processor 440 may determine a low blood glucose range (e.g., 90 to 140 mg/dL) past a given period of time (e.g., 1 hour) immediately after a meal based on 115 mg/dL. A high reference can be applied immediately after a meal because the blood glucose level rises, and a low reference can be applied during fasting because the blood glucose level falls due to the influence of insulin. The electronic device conforming to ones or more embodiments according to this document may determine the baseline blood glucose level and low blood glucose range reflecting the user's individual characteristics and circumstances. FIG. 7A and FIG. 7B illustrate a method in which the electronic device identifies a user's exercise situation and provides a notification to prevent a low blood glucose state before and after exercise according to ones or more embodiments.

The graph in FIG. 7A illustrates the blood glucose level over time for a normal person without diabetes. The horizontal axis may indicate time (hours), and the vertical axis may indicate blood glucose level (mg/100 mL). Referring to graph 700, the user's blood glucose level may rise for about 1 hour immediately after a meal, then insulin is secreted over about 1 to 2 hours, and the user's blood glucose level may fall to the normal range. This may be named a first section 701. Referring to graph 700, it can be seen that the user's blood glucose level temporarily falls during exercise as some of the glucose in the user's blood is used by the muscles. Thereafter, the user's blood glucose level may return to the normal range as glucagon is secreted. However, if a low blood glucose situation has been maintained before exercise, a hypoglycemic crisis may suddenly occur during exercise. Hence, the electronic device (e.g., electronic device 400 in FIG. 4) may check the user's blood glucose state at the start of exercise, warn of the risk of low blood glucose, and guide carbohydrate intake.

According to FIG. 7B, at operation 710, the electronic device 400 may present a guide screen asking whether to take exercise in response to a user input or a change in the user's state (e.g., heart rate change, location change), and may enter the exercise function based on the user input. As shown above in FIG. 7A, the user's blood glucose level may fall immediately after exercise, so the electronic device 400 may warn of the risk of low blood glucose in advance. When the electronic device 400 detects the start of exercise, it may provide a low blood glucose risk notification taking into consideration that the user's blood glucose level will fall due to subsequent exercise even if the user's blood glucose level is within the normal range.

At operation 720, the processor (e.g., processor 440 in FIG. 4) may identify the user's blood glucose level. Then, at operation 730, the processor 440 may check whether the user's blood glucose level is in a low blood glucose state. The low blood glucose state may be determined based on the baseline blood glucose level, and the baseline blood glucose level may be determined through the method of FIG. 6B to FIG. 6C above. If the user's blood glucose level does not belong to the low blood glucose range (operation 730—‘No’), at operation 735, the processor 440 may continuously proceed to the exercise. If the user's blood glucose level belongs to the low blood glucose range (operation 730—‘Yes’), at operation 740, the processor 440 may notify the user of a low blood glucose state and display a guide screen recommending carbohydrate intake on the display (e.g., display 420 in FIG. 4). The processor 440 may display a notification related to a low blood glucose state to the user even at the start of exercise other than during exercise. Additionally, when the processor 440 detects a situation where the user takes exercise, the processor 440 may take into consideration that the blood glucose level will fall during exercise. That is, the processor 440 may provide a low blood glucose warning notification and carbohydrate intake guide to the user even in a situation where the blood glucose level is higher than the low blood glucose range associated with the user's baseline blood glucose level.

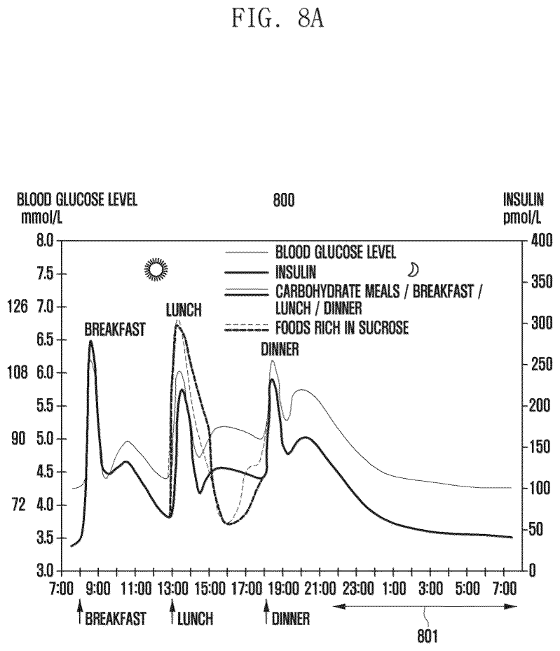

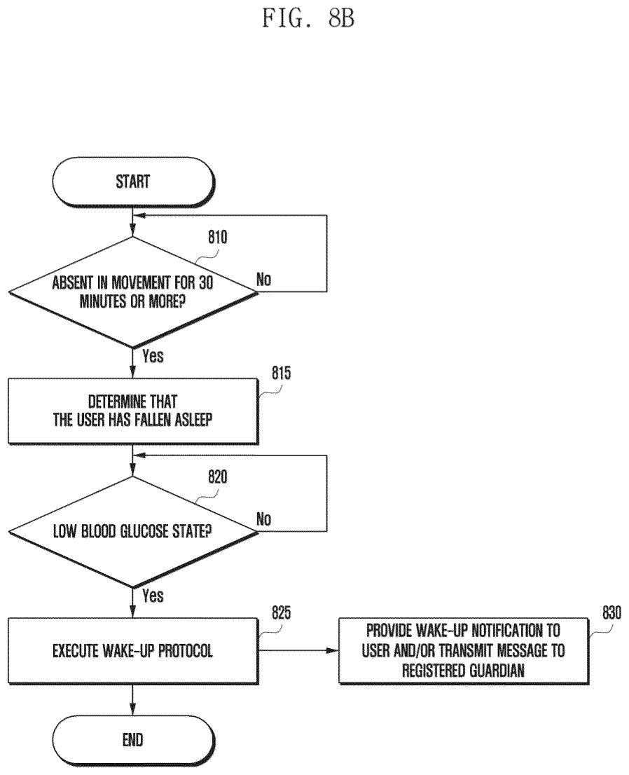

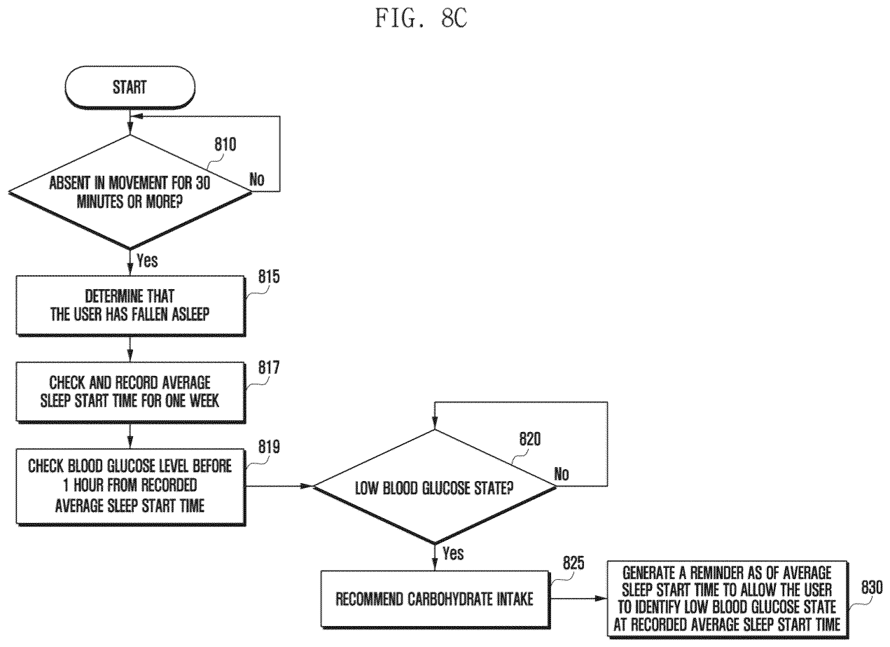

FIG. 8A, FIG. 8B, and FIG. 8C illustrate a method in which the electronic device identifies a user's sleep status and provides a notification to prevent a low blood glucose state before and during sleep according to ones or more embodiments.

Graph 800 in FIG. 8A illustrates the user's blood glucose level on an hourly basis. Changes in the blood glucose level immediately after meals, including breakfast, lunch, and dinner, have been previously described in FIG. 6A. Referring to graph 800 in FIG. 8A, it can be seen that the user's blood glucose level steadily falls during sleep (23:00˜07:00) (section 801) and reaches its lowest level when waking up in the morning (07:00).

As the brain activity decreases during sleep, the amount of glucose (or blood sugar) in the blood used as nutrients for the brain may also decrease. There is no problem when the user's blood glucose level before sleep is within the normal range, but problems may occur if sleep occurs with a blood glucose level being lower than the normal range. For example, the user may have difficulty in sleeping, may experience cold sweat after falling asleep, or may experience a severe headache after waking up. Further, if a low blood glucose state occurs repeatedly during sleep, the user may fall into a fainting state or coma. In particular, if the user is usually in a low blood glucose state or has a long evening fast, proper blood glucose management may be necessary.

In FIG. 8B, at operation 810, the processor 440 may determine whether the user has not moved for a given period of time (e.g., 30 minutes) or more. According to ones or more embodiments, the processor (e.g., processor 440 in FIG. 4) may use a sensor module (e.g., sensor module 410 in FIG. 4) to determine that the user is in a state just before sleep when there is no movement for a given period of time or more. The processor 440 may detect the user's movement within a given period of time (e.g., 30 minutes) and determine that the user is not sleeping. In this case, the processor 440 may continue to detect the user's movement. Upon determining that no user movement is detected for the given period of time (e.g. 30 minutes) or more, at operation 815, the processor 440 may determine that the user has entered a sleep state (or, falling asleep).

At operation 820, the processor 440 may determine whether the user is in a low blood glucose state. Upon determining that the user's blood glucose level is not in a low blood glucose state, the processor 440 may continue to monitor the user's blood glucose level without taking any special action. Alternatively, upon determining that the user's blood glucose level is in a low blood glucose state, at operation 825, the processor 440 may execute a wake-up protocol. The wake-up protocol may refer to at least one of the processes of preparing a sound notification, a vibration notification, or a screen notification to wake the user from a sleep state. Thereafter, at operation 830, the processor 440 may provide a wake-up notification to the user and/or transmit a message notifying the user's current low blood glucose state to the contact of a guardian set in advance. Additionally, the processor 440 may provide a guide for registering guardian's contact information to the user in order to transmit a message notifying the user's current low blood glucose state to the guardian's contact.

In FIG. 8C, at operation 810, the processor 440 may determine whether the user has not moved for a given period of time (e.g., 30 minutes) or more. According to ones or more embodiments, the processor (e.g., processor 440 in FIG. 4) may use a sensor module (e.g., sensor module 410 in FIG. 4) to determine that the user is in a state just before sleep when there is no movement for a given period of time or more. Upon detecting user's movement, the processor 440 may determine that the user is not sleeping and may continue to detect the user's movement. Upon determining that no user movement is detected for the given period of time (e.g. 30 minutes) or more, at operation 815, the processor 440 may determine that the user has entered a sleep state (or, falling asleep).

At operation 817, the processor 440 may check the user's sleep start time for a given period of time (e.g., one week) and record the average sleep start time on the memory (e.g., memory 450 in FIG. 4). The processor 440 may calculate the user's average sleep start time by accumulating recorded sleep start times of the user, and record the user's average sleep start time on the memory 450. Thereafter, at operation 819, the processor 440 may check the user's blood glucose level before a given time (e.g., 1 hour) from the recorded average sleep start time. The processor 440 may check the user's blood glucose level before a given time (e.g., 1 hour) from the recorded average sleep start time, and provide a warning notification to the user in advance taking into consideration that the blood glucose level falls during sleep. That is, even if the user's blood glucose level does not belong to the low blood glucose range based on the baseline blood glucose level, the processor 440 may provide an appropriate notification to the user by taking into account that the user's blood glucose level will fall during sleep.

At operation 820, the processor 440 may determine whether the user is in a low blood glucose state. Upon determining that the user's blood glucose level is not in a low blood glucose state, the processor 440 may continue to monitor the user's blood glucose level without taking a separate action. Alternatively, upon determining that the user's blood glucose level is in a low blood glucose state, at operation 840, the processor 440 may provide the user with a guide for carbohydrate intake. Additionally, the processor 440 may check the user's blood glucose level at the average sleep start time recorded on the memory 450. Upon identifying the user's low blood glucose state, the processor 440 may provide the user with a guide for carbohydrate intake again or may provide the user with a low blood glucose notification again.

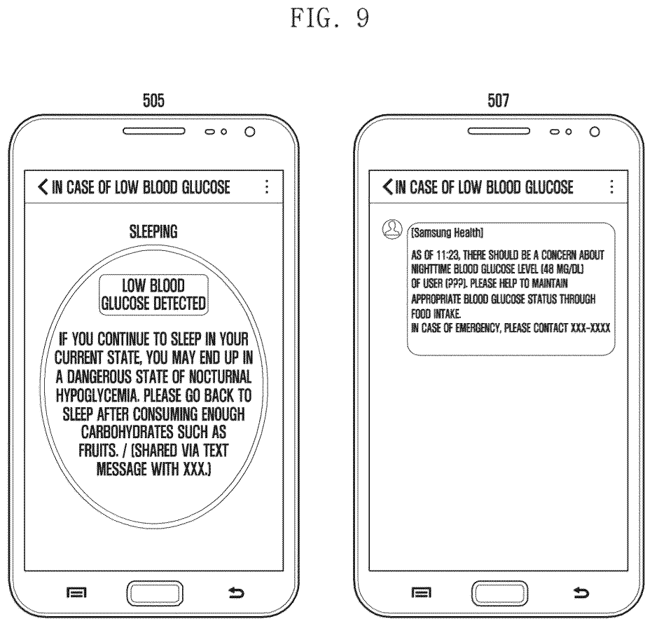

In operation 850, the processor 440 may generate a reminder to allow the user to identify the low blood glucose state. At this time, the processor 440 may control an operation to generate a reminder based on the recorded average sleep start time of the user. Next, in FIG. 9, a scheme for transmitting a notification to the user and/or another registered guardian will be described.

FIG. 9 is a diagram illustrating a situation in which an electronic device and an external device work together to provide a biometric information notification according to ones or more embodiments. In the following description, the electronic device may refer to the electronic device 400 in FIG. 4 or a wearable device, and a first external device 505 and a second external device 507 are assumed to be, but not limited to, a smartphone. In addition to a smartphone, the first external device 505 and the second external device 507 may include at least one of, for example, a laptop computer, a tablet computer, or a smart TV.

According to ones or more embodiments, the electronic device 400 may transmit and receive data to and from an external device 505 of the user by using a communication module (e.g., communication module 430 in FIG. 4). The electronic device 400 may transmit, to the external device 505, at least one of the user's eating habits information, glycemic index, risk level of eating habits, number of meals, warning notification based on determination of eating habits risk, or warning notification based on low blood glucose.

According to ones or more embodiments, the first external device 505 may refer to an equipment owned by the user. The second external device 507 may refer to an equipment owned by another user. According to ones or more embodiments, the electronic device 400 may determine a sleep state of the user by using the sensor module 410. The electronic device 400 may confirm the user's sleep state and confirm that the user's blood glucose level corresponds to a low blood glucose range, and provide a wake-up notification to the user through the first external device 505. Further, the electronic device 400 may confirm the user's sleep state and confirm that the user's blood glucose level corresponds to a low blood glucose range, and transmit a notification to the second external device 507 by using the communication module 430. In order to transmit a notification to the second external device 507, the first external device 505 may provide in advance a guide screen in which the user can add a contact to be used to send a notification when the user is sleeping.

According to ones or more embodiments, the external device 505 may include a cloud server, a personalization server, or a medical institution server. Additionally, the electronic device 400 may share the user's biometric information or eating habits information with an equipment of another person. For example, the electronic device 400 may transmit the user's eating habits information to a mobile terminal of a family member, or may transmit the user's eating habits information to a medical institution server or a personal health management server.

According to ones or more embodiments, the electronic device 400 may establish a communication connection with the external device 505. For example, the electronic device 400 may form a short-range communication link or a mobile communication link (e.g., 3G, 4G, 5G, etc.) with the external device 505. Short-range communication may include, but not limited to, Bluetooth, BLE (Bluetooth Low Energy), Wi-Fi Direct, UWB (ultra wideband), Zigbee, NFC (Near Field Communication), and/or Ant+.

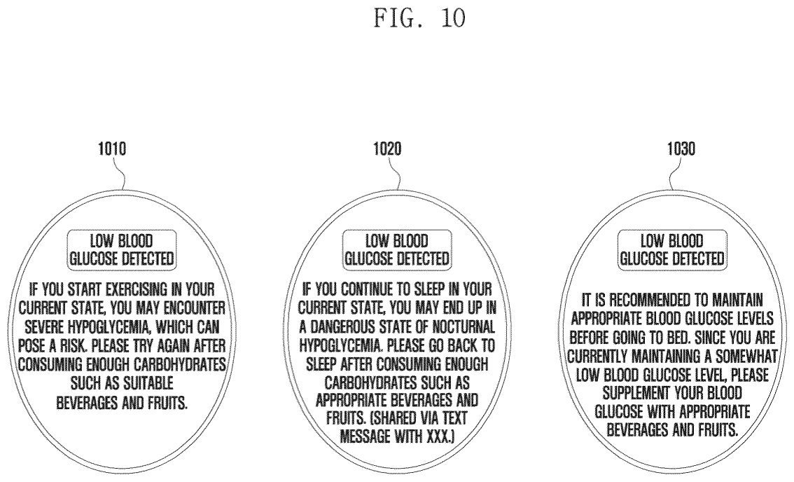

FIG. 10 illustrates an operation of the electronic device to display a notification depending on individual situations according to ones or more embodiments.

The electronic device (e.g., electronic device 400 in FIG. 4) may sense the user's state by using a sensor module (e.g., sensor module 410 in FIG. 4). The user's state that the electronic device 400 can sense may include, for example, at least one of exercise state, sleep state, or state just before sleep of the user.

According to ones or more embodiments, the electronic device 400 may use the sensor module 410 to confirm that the user is in an exercise state, check the user's blood glucose state at the start of exercise, warn of the risk of low blood glucose as shown in part 1010, and guide carbohydrate intake.

According to ones or more embodiments, the electronic device 400 may use the sensor module 410 to confirm that the user is sleeping, check the user's blood glucose state, warn of the risk of low blood glucose as shown in part 1020, and guide carbohydrate intake.

According to ones or more embodiments, the electronic device 400 may check the user's sleep start time for a week and store the average sleep start time in the memory (e.g., memory 450 in FIG. 4). The electronic device 400 may check the user's blood glucose level before a given time (e.g., 1 hour) from the average sleep start time stored in the memory 450. The electronic device 400 may provide a warning notification to the user as shown in part 1030 by confirming that the user's blood glucose level is in a low blood glucose range or taking into consideration that the blood glucose level falls during sleep. Further, the electronic device 400 may provide a guide that encourages the user to consume food containing appropriate nutrients (e.g., carbohydrates or sugar components).

According to ones or more embodiments, the electronic device may include a housing, a sensor module disposed inside the housing, a display, a memory, and a processor operably connected to the sensor module, the display, and the memory. The processor may monitor the blood glucose level of the user by using the sensor module, determine that the user is eating by confirming that the user's blood glucose level exceeds a specific level, record the time point of confirming that the user's blood glucose level exceeds a specific level and the user's blood glucose level at the time of confirmation in the memory, determine, based on blood glucose records between a first meal and a second meal, a first time point past a given time after the first meal and a second time point preceding a given time before the second meal, calculate the average value of blood glucose levels in the section between the first time point and the second time point, accumulate the calculated average value every day, calculate the baseline blood glucose level of the user by using a plurality of accumulated average value records, and determine a low blood glucose reference level of the user based on the calculated baseline blood glucose level.

According to ones or more embodiments, the processor may determine that the state of the user is a low blood glucose state by confirming that the user's blood glucose level is below the low blood glucose reference level, and control the display to display a notification indicating an occurrence of hypoglycemia, wherein the user's low blood glucose reference level may include approximately 60% of the calculated baseline blood glucose level of the user.

According to ones or more embodiments, the processor may monitor the blood glucose level of the user by using the sensor module, periodically present a guide screen to the user to allow the user to select whether the user is eating, confirm that the user is eating based on the user's response, determine a third time point past a given time after the confirmation time point, measure the blood glucose level at regular intervals from the third time point, calculate the average value of the measured blood glucose levels, accumulate the calculated average value every day, calculate the baseline blood glucose level of the user by using a plurality of accumulated average value records, and determine a low blood glucose reference level of the user based on the calculated baseline blood glucose level of the user.

According to ones or more embodiments, the processor may determine that the first meal is over when the blood glucose level falls in excess of a given time on the blood glucose level records, and may determine that the second meal has started when the blood glucose level rises again in excess of a given time on the blood glucose level records.

According to ones or more embodiments, the processor may use the sensor module to determine that the user is in a state just before sleep when the user does not move for a given period of time or more.

According to ones or more embodiments, the processor may confirm that the user is sleeping through the sensor module, and if the blood glucose level measured at regular intervals while the user is sleeping is below the user's low blood glucose reference level, it may generate a notification to encourage the user to wake up.

According to ones or more embodiments, the processor may confirm that the user is sleeping through the sensor module, and if the blood glucose level measured at regular intervals while the user is sleeping is below the user's low blood glucose reference level, it may transmit a notification to a preset external device.

According to ones or more embodiments, the processor may determine the user's sleep state and sleep time by using the sensor module, and store the user's average sleep start time of falling asleep in the memory.

According to ones or more embodiments, the processor may check the user's blood glucose level before a given time from the user's average sleep start time of falling asleep, and may generate a notification containing information encouraging appropriate food intake when the user's blood glucose level falls within a given range compared to the user's low blood glucose reference level.

According to ones or more embodiments, the processor may check the user's blood glucose level in response to detection of a user's exercise state through the sensor module or user input for an exercise state.

According to ones or more embodiments, based on the user's exercise state, the processor may generate a low blood glucose risk notification when the user's blood glucose level falls within a given range compared to the low blood glucose reference level.

According to ones or more embodiments, a biometric information notification method of an electronic device may include: monitoring the user's blood glucose level by using a sensor module; determining that the user is eating by confirming that the user's blood glucose level exceeds a specific level, and recording at least one of the time point of confirming that the user's blood glucose level exceeds a specific level or the user's blood glucose level at the time of confirmation in the memory; determining, based on blood glucose records between a first meal and a second meal, a first time point past a given time after the first meal and a second time point preceding a given time before the second meal, and calculating the average value of blood glucose levels in the section between the first time point and the second time point; accumulating the calculated average value every day; calculating the baseline blood glucose level of the user by using a plurality of accumulated average value records; and determining a low blood glucose reference level of the user based on the calculated baseline blood glucose level.

According to ones or more embodiments, the biometric information notification method of the electronic device may further include: determining that the state of the user is a low blood glucose state by confirming that the user's blood glucose level is below the low blood glucose reference level; and displaying a notification indicating an occurrence of hypoglycemia by using a display, wherein the user's low blood glucose reference level may correspond to approximately 60% of the calculated baseline blood glucose level of the user.

According to ones or more embodiments, the biometric information notification method of the electronic device may further include: monitoring the blood glucose level of the user by using the sensor module; periodically presenting a guide screen to the user to allow the user to select whether the user is eating, and confirming that the user is eating based on the user's response; determining a third time point past a given time after the confirmation time point; measuring the blood glucose level at regular intervals from the third time point, and calculating the average value of the measured blood glucose levels; accumulating the calculated average value every day, and calculating the baseline blood glucose level of the user by using a plurality of accumulated average value records; and determining a low blood glucose reference level of the user based on the calculated baseline blood glucose level of the user.

According to ones or more embodiments, the biometric information notification method of the electronic device may further include determining that the user is in a state just before sleep in response to confirming that the user does not move for a given period of time or more by using the sensor module.

According to ones or more embodiments, the biometric information notification method of the electronic device may further include: confirming that the user is sleeping through the sensor module; generating a notification for encouraging the user to wake up in response to confirming that the blood glucose level measured at regular intervals while the user is sleeping is below the user's low blood glucose reference level; and transmitting the notification to a preset external device.

According to ones or more embodiments, the biometric information notification method of the electronic device may further include determining the user's sleep state and sleep time by using the sensor module and storing the user's average sleep start time of falling asleep in the memory.

According to ones or more embodiments, the biometric information notification method of the electronic device may further include: checking the user's blood glucose level before a given time from the user's average sleep start time of falling asleep; and generating a notification containing information encouraging appropriate food intake when the user's blood glucose level falls within a given range compared to the user's low blood glucose reference level.

According to ones or more embodiments, the biometric information notification method of the electronic device may further include checking the user's blood glucose level in response to detection of a user's exercise state through the sensor module or user input for an exercise state.

According to ones or more embodiments, the biometric information notification method of the electronic device may further include: determining whether the user is taking exercise in response to detection of a user's exercise state through the sensor module or user input for an exercise state; and generating a low blood glucose risk notification in response to confirming that the user is taking exercise and that the user's blood glucose level falls within a given range compared to the low blood glucose reference level.

What is claimed is:

1. An electronic device comprising:
a housing;
a sensor module disposed inside the housing;
a display;
a memory; and
a processor operably connected to the sensor module, the display, and the memory,
wherein the processor is configured to:
monitor a blood glucose level of a user by using the sensor module;
determine that the user is eating based on confirming that the blood glucose level exceeds a specific level;
record, in the memory, both a time point of confirming that the blood glucose level exceeds the specific level and the blood glucose level at the time point;
determine, based on blood glucose records between a first meal and a second meal, a first time point past a first given time after the first meal and a second time point preceding a second given time before the second meal;
determine an average value of blood glucose levels between the first time point and the second time point;
obtain a plurality of accumulated average value records by accumulating the average value daily;
determine a baseline blood glucose level of the user by using the plurality of accumulated average value records; and
determine a low blood glucose reference level of the user based on the baseline blood glucose level.

2. The electronic device of claim 1, wherein the processor is further configured to:
determine that the user is in a low blood glucose state based on confirming that the blood glucose level of the user is below the low blood glucose reference level; and
control the display to display a notification indicating an occurrence of hypoglycemia, and
wherein the low blood glucose reference level is 60% of the determined baseline blood glucose level of the user.

3. The electronic device of claim 2, wherein the processor is further configured to:
control the display periodically display a guide screen to the user, wherein the guide screen is configured to receive a user selection confirming whether the user is eating;
determine a third time point past a third given time after receiving the user selection confirming whether the user is eating;
measure the blood glucose level at regular intervals from the third time point;
determine a second average value of the measured blood glucose levels at the regular intervals from the third time point;
accumulate a plurality of second average values, including the second average value, daily;
determine a second baseline blood glucose level of the user by using the plurality of second average values; and
determine a second low blood glucose reference level of the user based on the second baseline blood glucose level of the user.

4. The electronic device of claim 1, wherein the processor is further configured to:
determine that the first meal has ended based on the blood glucose level falling on blood glucose level records; and
determine that the second meal has started based on the blood glucose level rising again on the blood glucose level records.

5. The electronic device of claim 1, wherein the processor is further configured to, based on no user movement being detected through the sensor module for a preset time or more, determine that the user is in a state just before sleep.

6. The electronic device of claim 5, wherein the processor is further configured to:
confirm, through the sensor module, that the user is sleeping; and
generate a notification, to the user to wake up, based on a blood glucose level measured at regular intervals while the user is sleeping being below the low blood glucose reference level.

7. The electronic device of claim 5, wherein the processor is further configured to:
confirm, through the sensor module, that the user is sleeping; and
transmit a notification, to a preset external device, based on a blood glucose level measured at regular intervals while the user is sleeping being below the blood glucose reference level.

8. The electronic device of claim 1, wherein the processor is further configured to:
determine, by using the sensor module, a sleep state of the user and a sleep time of the user; and
store, in the memory, an average sleep start time of falling asleep.

9. The electronic device of claim 8, wherein the processor is further configured to:
check the blood glucose level at a preset time before the average sleep start time of falling asleep; and
generate a notification, containing information on food intake, based on the blood glucose level falling within a given range compared to the low blood glucose reference level.

10. The electronic device of claim 1, wherein the processor is further configured to, based on detection of an exercise state of the user through at least one of the sensor module and a user input indicating an exercise, check the blood glucose level state.

11. The electronic device of claim 10, wherein the processor is further configured to generate a low blood glucose risk notification based on the exercise state and the blood glucose level falling within a given range compared to the low blood glucose reference level.

12. A method for an electronic device to notify biometric information, the method comprising:
monitoring, by using a sensor module, a blood glucose level of a user;
based on confirming that the blood glucose level exceeds a specific level, determining that the user is eating;
recording, in a memory, at least one of a time point of confirming that the blood glucose level exceeds a specific level or the blood glucose level at the time point;
determining, based on blood glucose records between a first meal and a second meal, a first time point past a first given time after the first meal and a second time point preceding a second given time before the second meal;
determining an average value of blood glucose levels between the first time point and the second time point;
obtaining a plurality of average value records by accumulating the average value daily;
determining a baseline blood glucose level of the user by using the plurality of average value records; and
determining a low blood glucose reference level of the user based on the baseline blood glucose level of the user.

13. The method of claim 12, further comprising:
based on confirming that the blood glucose level is below the low blood glucose reference level, determining that the user is in a low blood glucose state; and
displaying, by using a display, a notification indicating an occurrence of hypoglycemia,
wherein the low blood glucose reference level is 60% of the baseline blood glucose level of the user.

14. The method of claim 12, further comprising:
periodically displaying a guide screen to the user, wherein the guide screen is configured to receive a user selection confirming whether the user is eating;
determining a third time point past a third given time after receiving the user selection confirming whether the user is eating;
measuring the blood glucose level at regular intervals from the third time point;
determining a second average value of the measured blood glucose levels at the regular intervals from the third time point;
accumulating a plurality of second average values, including the second average value, daily;
determining a second baseline blood glucose level of the user by using the plurality of second average values; and
determining a second low blood glucose reference level of the user based on the second baseline blood glucose level of the user.

15. The method of claim 12, further comprising, based on confirming that no user movement is detected through the sensor module for a preset time or more, determining that the user is in a state just before sleep.
